(19) United States
(12) Patent Application Publication
(10) Pub. No.: US 2022/0280699 A1
(43) Pub. Date: Sep. 8, 2022

(54) NANOCOATINGS AND METHODS FOR FABRICATING AN INTRACARDIAC ECHOCARDIOGRAPHY ULTRASOUND TRANSDUCER

(71) Applicant: St. Jude Medical, Cardiology Division, Inc., St. Paul, MN (US)

(72) Inventor: Xiaoping Guo, Eden Prairie, MN (US)

(21) Appl. No.: 17/684,854

(22) Filed: Mar. 2, 2022

Related U.S. Application Data
(60) Provisional application No. 63/156,072, filed on Mar. 3, 2021.

(51) Int. Cl. A61L 29/12; A61L 29/08; A61L 29/10; A61L 29/14; C08L 83/04

(52) U.S. Cl. CPC A61L 29/126; A61L 29/085; A61L 29/106; A61L 29/14; C08L 83/04

(57) ABSTRACT

An intracardiac echocardiography catheter includes a shaft and an ultrasound transducer at a distal end of the shaft. The ultrasound transducer includes an outer polymeric encapsulant layer and a nanocoating applied to the outer polymeric encapsulant layer. The nanocoating is configured to provide increased surface lubricity and self-cleaning properties to the ultrasound transducer.

### CROSS-REFERENCE TO RELATED PATENT APPLICATIONS

This application claims the benefit of and priority to U.S. Provisional Patent Application 63/156,072, filed Mar. 3, 2021, which is hereby incorporated by reference in its entirety.

### BACKGROUND

The present disclosure relates to coatings for an ultrasound transducer. More particularly, the present disclosure relates to a nanocoating for a distal ultrasound transducer of an intracardiac echocardiography (ICE) catheter.

A typical ICE catheter includes a deflectable distal ultrasound transducer. Due to its distal deflectability, an ICE catheter allows for omni-directional ultrasound imaging to visualize intracardiac anatomy, blood flow and devices inside the heart. This helps eliminate unnecessary fluoroscopy (X-ray) exposure to the patient and physician. For example, a deflectable ICE catheter allows a clinician to explore detailed structure inside the heart, identify the exact location of the catheter tip, and thus perform precision transcatheter EP ablation procedures for treating atrial fibrillation (AF), ventricular tachycardias and the pulmonary vein. Beyond its common uses in cardiac catheterization laboratories and EP, an ICE catheter system has also found its increasing applications in structural heart disease repairs. For example, an ICE catheter, when connected to a compatible control unit, provides a real-time imaging modality similar to intravascular ultrasound (IVUS), and can be utilized to image the left atrial appendage (LAA), to assist in the closure of patent foramen ovale (PFO) and atrial septal defects (ASD) and visualization of the fossa ovalis, and to support the transcatheter valve replacements, and other structural heart disease repairs.

The ultrasound transducer of an ICE catheter transmits the activating ultrasound waves directly to, and receives the echo waves directly from, the heart anatomy. The transducer, therefore, makes frequent contact with the heart tissue, during ultrasound imaging in versatile left-to-right and/or anterior-to-posterior configurations of the transducer during the ultrasound imaging procedure. This imposes varying sliding frictions and pressures against the tissue wall of the heart by the transducer, which may be undesirable.

In addition, the ultrasound transducer of an ICE catheter is an integral assembly that consists of various essential functional components and is entirely enclosed by a polymeric shell or an outer polymeric encapsulant layer having the desirable physical softness and requisite acoustic properties of material. In order to achieve consistent performances of ultrasound imaging, the external surfaces of the outer polymeric encapsulant layer should be free of any surface contaminants that may disrupt the transmission and receiving of acoustic energy between the heart structure and the transducer.

In consideration of the foregoing, the present disclosure relates to a coating applied to the external surfaces of the outer polymeric encapsulant layer for the ultrasound transducer of an ICE catheter.

### SUMMARY OF THE INVENTION

One embodiment relates to an intracardiac echocardiography catheter including a shaft and an ultrasound transducer at a distal end of the shaft. The ultrasound transducer includes an outer polymeric encapsulant layer and a nanocoating applied to the outer polymeric encapsulant layer. The nanocoating is configured to provide increased surface lubricity and self-cleaning properties to the ultrasound transducer.

Another embodiment relates to an organosilane nanocoating dispersion for coating an intracardiac echocardiography ultrasound transducer including at least one solvent, at least one reactive organosilane, at least one nano-sized particle substance, and optionally but preferably, a catalyst.

Another embodiment relates to a method of manufacturing an ultrasound transducer that includes manufacturing an ultrasound transducer having an outer polymeric encapsulant layer and cleaning an outer polymeric surface of the ultrasound transducer. The method further includes applying a nanocoating to the ultrasound transducer, removing excess nanocoating from the ultrasound transducer, allowing the nanocoating on the outer polymeric encapsulant layer of the ultrasound transducer to hydrolyze, and curing the nanocoating applied on the ultrasound transducer.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a schematic drawing of an intracardiac echocardiography (ICE) catheter system according to some embodiments;

FIG. 2A is a schematic drawing of an ultrasound transducer of an ICE catheter, in a cross-sectional side view, according to some embodiments;

FIG. 2B is schematic drawing of the ultrasound transducer of the ICE catheter of FIG. 2A, in a cross-sectional end view, according to some embodiments;

FIG. 2C is a schematic drawing of the ultrasound transducer of the ICE catheter of FIG. 2A, in a side-view, according to some embodiments;

FIG. 3 depicts molecular structures for exemplary monomeric organosilanes relevant to some embodiments of a coating for the ultrasound transducer;

FIG. 4 is a schematic depiction of the hydrolysis-condensation cure mechanism in general silane and silicone chemistry relevant to some embodiments of a coating for the ultrasound transducer;

FIG. 5 is a chemical illustration of various exemplary additives for the coating material according to some embodiments;

FIG. 6 is a process flow chart of a method for coating an ultrasound transducer of an ICE catheter according to some embodiments;

FIG. 7 is a chemical illustration of a functionalized poly(hedral oligomeric silsesquoxane) (POSS) nanoparticle comprising trisilanol group, relevant to some embodiments of a coating for the ultrasound transducer.

### DETAILED DESCRIPTION

Some exemplary embodiments of the present disclosure relate to organosilane nanocoating dispersion formulations and effective manufacturing methods for modifying the exposed surfaces of an outer polymeric encapsulant layer which fully and integrally encloses various constituent functional components of an ultrasound transducer. The exposed surfaces of the outer polymeric encapsulant layer may be herein referred to as “the transducer surfaces” for simplicity. The modified transducer surface by such silane or organosilane nanocoating exhibits high surface lubricity to minimize damage to the heart tissue. Functionally, the nanocoating film applied and cured on the active portion of the outer polymeric encapsulant layer, namely the matching material layer, may also act as the acoustic lens for the ultrasound transducer. Therefore, in addition to its surface lubricity, such an acoustic lens possesses inherent surface superhydrophobicity, thus imparting self-cleaning capacity and enhancing performance consistency for the ultrasound transducer.

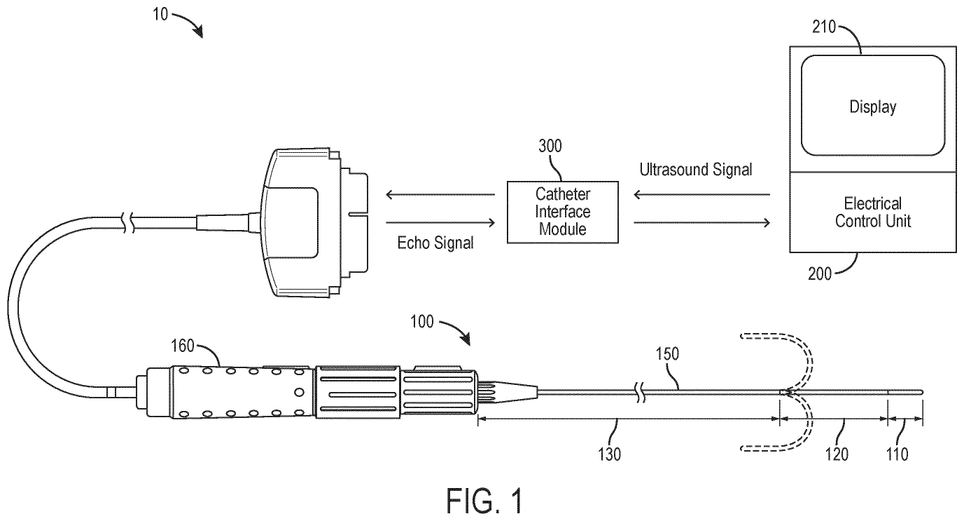

As schematically shown in FIG. 1, an exemplary intracardiac echocardiography (ICE) catheter system 10 includes an ICE catheter 100 having a distal ultrasound transducer 110 (or probe), an electronic control unit (ECU) 200, and a catheter interface module 300. The ECU 200 electrically controls various imaging functions provided by the distal ultrasound transducer 110 of the catheter 100, including, but not limited to, sending the activating, ultrasound electrical signals to, and receiving and recording the resultant echo signal data from, the ultrasound transducer 110. Based on the received echo signal data, the ECU 200 also renders the ultrasound image on its display 210. The catheter interface module 300 isolates an electrical power supply of the ICE system 10 from the patient and only transmits the isolated, activating ultrasound signals to, and the echo signals from, the ultrasound transducer 110 mounted at the distal end of the ICE catheter 100.

Referring still to FIG. 1, the ICE catheter 100 includes a catheter shaft 150 having three sections, namely a distal section including a distal ultrasound transducer 110 imparting the essential ultrasound imaging capacities, a deflectable section 120 allowing for distal deflectability and steerability of catheter shaft 150, and a proximal body section 130 extending from a handle 160, providing column strength and torqueability of catheter shaft 150. As such, the catheter 100 can navigate through the tortuous vascular system and reach the desirable sites within the complex heart anatomy. This construction also allows the distal ultrasound transducer 110 to deflect in left-to-right or anterior-to-posterior configurations for omni-directional ultrasound imaging on the heart anatomy by controlling the catheter handle 160 to manipulate relevant deflection mechanisms of the catheter shaft 150.

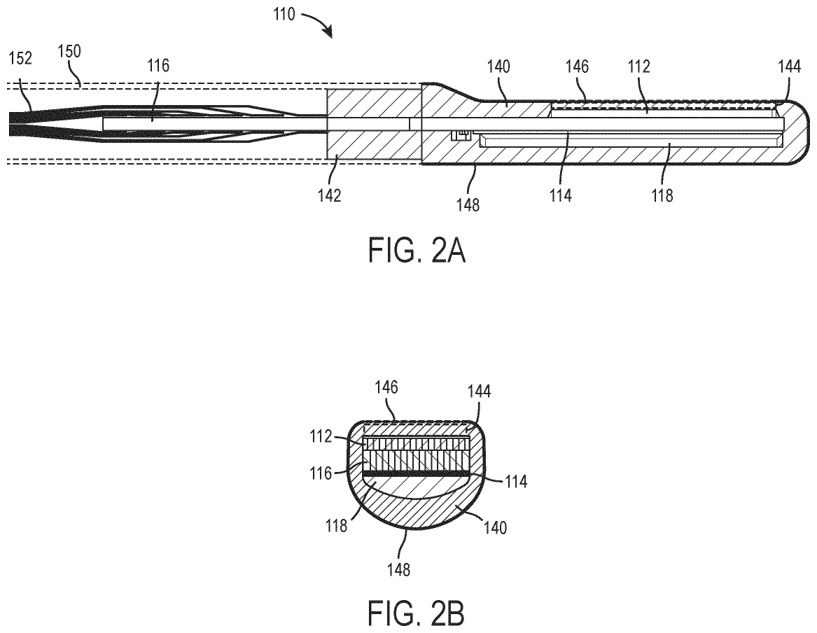

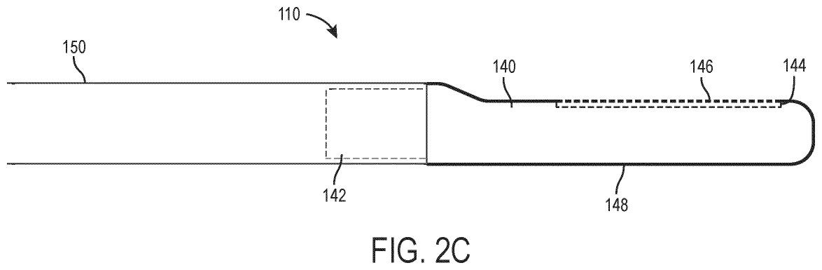

FIGS. 2A-2B show side and end cross-sectional views, respectively, of the ultrasound transducer 110. As shown in FIGS. 2A-2B, in an exemplary embodiment, the ultrasound transducer 110 (or probe) mounted at the distal end of the ICE catheter 100 generally includes an acoustic stack 112. The acoustic stack 112 is comprised of an array of piezoelectric elements. In some embodiments, the acoustic stack 112 is made of a piezoelectric material selected from relatively rigid PZT piezoceramics (such as PZT-5A, PZT-5H, etc.), polycrystalline PMN-PT and PZN-PT, etc. The acoustic stack 112 is placed onto the application specific integrated circuitry (ASIC) 114 of a multilayered printed circuit board (PCB) 116. In some embodiments, the acoustic stack 112 is physically adhered and electrically connected to the PCB 116. The PCB 116 is further electrically connected to a bundle of conductor wires 152, which extends along the central lumen of catheter shaft 150 (see FIG. 1), and is cabled within and through the catheter handle 160 to electrically couple with the catheter interface module 300. As such, the activating, ultrasound electrical signals from the ECU 200 can be reliably applied to the acoustic stack 112 of the ultrasound transducer 110 to generate mechanical waves.

To prevent excessive vibration within the acoustic stack 112 and allow the ultrasound waves to transmit in the direction towards the heart anatomy and at a relatively short pulse length for improving axial resolution of ultrasound imaging, a backing material layer 118 is physically located behind the acoustic stack 112 (which is physically and electrically integrated with the PCB 116 and ASIC 114). The backing material layer 118 should have an effective acoustic absorption or damping capacity, possess a relatively high acoustic impedance that is similar to the acoustic stack 112, and be able to firmly adhere to the acoustic stack 112 and ASIC 114. For these purposes, the backing material layer 118 may be made of filled polymer composites, such as relatively rigid, thermosetting epoxy/urethane composites filled with heavy metal particles (e.g. tungsten, barium sulfate, etc.). In other embodiments, a relatively rigid solid block machined of a metallic material, such as steel, copper and brass, titanium, etc., can be also used as a good backing material layer 118. Such a block can serve as the rear electrode for the ASIC 114 of the PCB 116, while the front electrodes for the ASIC 114 of the PCB 116 may be physically and electrically pre-integrated with individual piezoelectric elements of the acoustic stack 112.

Ultrasonic waves transmitting from the piezoelectric elements of the acoustic stack 112 may be largely reflected off targeted heart anatomy because of considerable differences in acoustic impedance (i.e. the resistance of material to the passage of sound waves) between the acoustic stack 112 of the transducer 110 and the heart anatomy. To minimize such reflections and maximize the wave transmission into the internal structure of the heart anatomy for ultrasound imaging at relatively high sensitivity, a matching material layer 144, which functions as the active portion of an outer polymeric encapsulant layer 140, is applied directly on the top side of the acoustic stack 112 at a controlled thickness.

As shown in FIG. 2C, the outer polymeric encapsulant layer 140 fully encloses various constituent components (e.g. backing material layer 118, ASIC 114, acoustic stack 112, and PCB 116) of the ultrasound transducer 110, and makes up the cylindrical extension 142 for physical integration with a hollow catheter shaft 150. The matching material layer 144 is the portion of the outer polymeric encapsulant layer 140 which is positioned on the top side of the acoustic stack 112, and is therefore the active portion of the outer polymeric encapsulant layer 140. The outer polymeric encapsulant layer 140 may be formed, along with the cylindrical extension 142 via a mold casting or liquid molding process, followed by thermal curing under ambient condition or at elevated temperatures lower than 70° C., thus forming the outer polymeric encapsulant layer 140 in entirety. As shown in FIG. 2C, the catheter shaft 150 and the ultrasound transducer 110 can be firmly adhered together by using the lap-joined structure located at the cylindrical extension 142 via adhesive bonding or other applicable joining method.

To allow for wave transmission from the acoustic stack 112 to the heart anatomy with the maximal transmission and the minimal reflection, the matching material layer 144 of the ultrasound transducer 110, as the active portion of the outer polymeric encapsulant layer 140, has an intermediate acoustic impedance (Zₘ) higher than that of the heart tissue (e.g. Zₕ˜1.64 MRayl), but considerably lower than the acoustic stack 112 (and backing material layer 118) of the transducer 110 (e.g. Zₚ˜30 to 35 MRayl). More specifically, the acoustic impedance for the matching material layer 144 should satisfy the equation Zₘ=\sqrt{ZₚZₕ}. Noting that acoustic impedance of material Z=\sqrt{Bρ} where B is bulk modulus or material stiffness and is material density, the acoustic impedance for the matching material layer 144 (and the outer polymeric encapsulant layer 140 in entirety) may be adjusted by using common rubber compounding techniques with the addition of heavy inorganic particles (e.g. barium sulfate, etc.). The incorporation of heavy inorganic filler would increase material's density and bulk modulus, and thus increase the acoustic impedance for the matching material layer 144, while the feel of material softness would not be affected. Accordingly, the matching material layer 144 would be able to effectively transmit the activating ultrasound waves from the acoustic stack 112 of the transducer 110 to the structure of the heart anatomy with relatively small reflection by the heart tissue, thus imparting adequate sensitivity for ultrasound imaging.

Furthermore, in cast molding, the thickness of the matching material layer 144, as the active portion of the outer polymeric encapsulant layer 140 that considerably affects the acoustic performance of the transducer 110, is well controlled in order to optimize the activating ultrasound energy transmission within the material matching layer 144 for the given frequency bandwidths of ultrasound imaging as designed for the ICE catheter system 10. In some embodiments disclosed herein, an optimal thickness of the matching material layer 144 is controlled as one quarter of the wavelength of the ultrasound waves transmitting within the material matching layer 144. Except for the matching material layer 144, there are no specific requirements on the acoustic properties or thickness for the other portions, including the cylindrical receptacle portion 142, of the outer polymeric encapsulant layer 140. Therefore, in consideration of manufacturability, the entire ultrasound transducer 110 is fully and integrally enclosed using the same polymeric encapsulant material as required for the matching material layer 144.

Based on the above functional requirements of the matching material layer 144, a soft, atraumatic thermoplastic elastomer or thermosetting rubber is preferably selected for the outer polymeric encapsulant layer 140. Furthermore, in consideration of thermal sensitivity of the pre-integrated PCB 116 and ASIC 114 with the acoustic stack 112, a reactive thermosetting rubber system with good liquid fluidity and low-temperature curability at temperatures near or less than 70° C. (e.g. urethane rubber or silicone rubber etc.) is preferably utilized for making the matching material layer 144, as part of the outer polymeric encapsulant layer 140 in entirety. A two-part silicone rubber system cured by platinum-catalyzed hydrosilylation or addition curing is preferred due to its inherent hydrophobicity, biocompatibility, thermo-physical and thermo-chemical stability, and liquid processability. For example, a 25 to 55 wt. % silica-filled, liquid silicone rubber system, e.g. two-part Dow Corning Silastic™ MDX4-4120 RTV silicone elastomer, Dow Corning Silastic™ RTV 4130-J, etc., may be used to form the matching material layer 144 at a controlled thickness, while the other remaining portions of the outer polymeric encapsulant layer 140 have no specific thickness requirement.

As discussed above and referring still to FIGS. 2A-2C, the ultrasound transducer 110 includes various functional components, which are fully enclosed by the same, soft/atraumatic, outer polymeric encapsulant layer 140, namely a silicone rubber material, such that the ultrasound transducer 110 has a matching material layer 144 at a controlled layer thickness for serving as the acoustic lens 146 and imparting high imaging performance (e.g. high sensitivity and high axial resolution). However, the outer surfaces of the outer polymeric encapsulant layer 140 lack surface lubricity and self-cleaning capacity. Instead, soft silicone rubber material exhibits inherent material stickiness and is prone to attracting foreign materials.

The material stickiness of the outer polymeric encapsulant layer 140 of the ultrasound transducer 110 can impose varying sliding frictions and pressures against the tissue wall of the heart as the ultrasound transducer 110 makes frequent contact with the heart tissue during the ultrasound imaging procedure. Furthermore, in order to achieve consistent performance for ultrasound imaging, the external surface of the matching material layer 144 of the ultrasound transducer 110, should be free of any surface contaminants that may disrupt the transmission and receipt of acoustic energy between the heart structure and the transducer 110, but the soft silicone rubber material of the outer polymeric encapsulant layer 140 is prone to attracting foreign materials. Therefore, in consideration of the above, a surface modification of the silicone rubber-encapsulated ultrasound transducer 110 is needed to improve surface lubricity and impart self-cleaning capacity. To this end, a nanocoating 148 is applied to the outer surfaces of the outer polymeric encapsulant layer 140, including the material matching layer 144, and is described in detail below.

According to the present invention, surface lubricity (i.e. the property of a material having a low coefficient of friction (COF) against itself and other objects in direct contact), self-cleaning properties, as well as adhesive bondability to the catheter shaft, is achieved by applying polymer hydrophobic coatings to the outer surface of the outer polymeric layer 140 of the ultrasound transducer 110.

A hydrophobic polymer coating, as characterized by the water contact angle 90°<<0<180° of its surface, is generally unaffected by the wetting of water, and thus, it is physically and mechanically stable, regardless of its dry and wetted states. Accordingly, such hydrophobic polymer coating material, when applied directly onto the matching material layer 144 to act as the acoustic lens 146 of the ultrasound transducer 110, would generally have the stable acoustic impedance (Z₁). Since the coating thickness is generally at submicron scale, the physically stable coating material as the acoustic lens 146 of the ultrasound transducer 110 would not affect the activating ultrasound wave transmission as long as its acoustic impedance Z₁ is comparable to Zₘ.

Furthermore, superhydrophobic polymer coatings, as characterized by very high water-contact-angles in the range of 150 to 180°, would exhibit exceptionally high water-repellency (or nearly non-wettability by water), thus further imparting some unique surface properties, such as anti-fouling and self-cleaning, etc. Therefore, to achieve the desirable surface properties for the ultrasound transducer 110, the development of lubricious, superhydrophobic polymer coatings for the matching material layer 144 is of particular interest to make the high-performance acoustic lens 146 for the ultrasound transducer 110. In consideration of manufacturability and the required surface lubricity of the ultrasound transducer 110, in some embodiments, the same coating is applied to the entire outer polymeric encapsulant layer 140, thus forming the thin coating layer 148 onto the exposed silicone surfaces of the ultrasound transducer 110.

The present disclosure relates specifically to various lubricious superhydrophobic polymer nanocoating formulations, namely organosilane or silane nanocoatings, with functional nanofillers to be used for the lubricious coating surface 148 of the ultrasound transducer 110 which makes up the high-performance acoustic lens 146. These organosilane or silane nanocoatings would inherently exhibit chemical compatibility and adherence to the outer polymeric encapsulant layer 140 comprising the matching layer material 144 without requiring chemical activation of the silicone rubber. Relevant coating technology, formulations and processes are described herein.

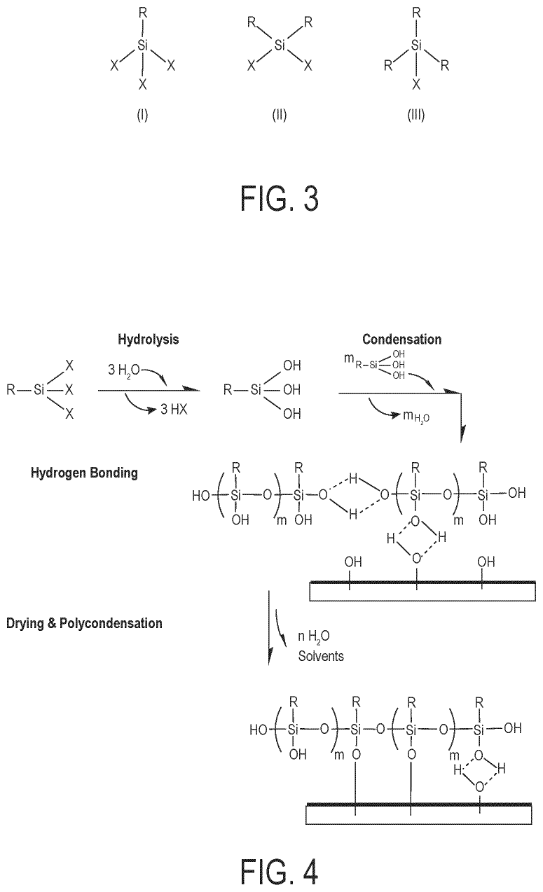

According to silane chemistry, reactive organosilanes are various silicon-based monomers comprising at least one “Si—C” bond due to an organofunctional group (R) and at least one hydrolytically-sensitive center due to a hydrolyzable group (X) that can react with moisture or hydroxylated substrate surfaces as commonly seen in some inorganic materials (e.g. glasses, metals, silica, titanium dioxide, etc.). The molecular structures for some typical monomeric organosilanes are shown in FIG. 3, where R denotes the same or different organofunctional moiety independently selected from linear and branched alkyl, cycloalkyl, aryl, and alkyl aryl groups having 1 to 18 carbon atoms and from allyl (CH₂═CH—CH₂—R′—) or vinyl (CH₂═CH—), acryloyl (CH₂═CH—C(═O)—R′—), and methacryloyl (CH₂═C(—CH₃)—C(═O)—R′—) groups, and combinations thereof, and where R′ is alkyl group. X represents the same or different hydrolyzable moiety independently selected from hydrogen, halogen, hydroxy, alkoxy, acyloxy, amine group, and combinations thereof.

Most monomeric organosilanes for polymer coating and surface modification have one to three organofunctional moieties (R) and one to three hydrolyzable moieties (X) according to Formula (I), (II) and (III) illustrated in FIG. 3. The underlying hydrolysis-condensation cure mechanism in general silane and silicone chemistry is shown schematically in FIG. 4. As shown, upon exposure to a hydroxylated substrate surface, or water purposely added to relevant organosilane coating solution/dispersion, or moisture from the atmosphere, the constituent hydrolyzable moieties (X) of a monomeric organosilane are first hydrolyzed to form highly reactive multifunctional silanol-containing species (R—Si—(OH)₃) with the release of relevant byproduct (HX), followed by condensation to the silanol-containing oligomers with the concomitant release of water (H₂O) as byproduct. These oligomers then hydrogen bond with themselves and/or with the hydroxylated substrate surface. Finally, during drying or curing under ambient condition or at elevated temperature, covalent siloxane (—Si—O—Si—) bonds among oligomeric molecules, which are present on the substrate and/or within the substrate sub-surface, would form via the concurrent loss of both water and solvents, thus leading to the formation of three dimensional, chemically crosslinked hydrophobic, siloxane polymer film of coating adhered to the substrate.

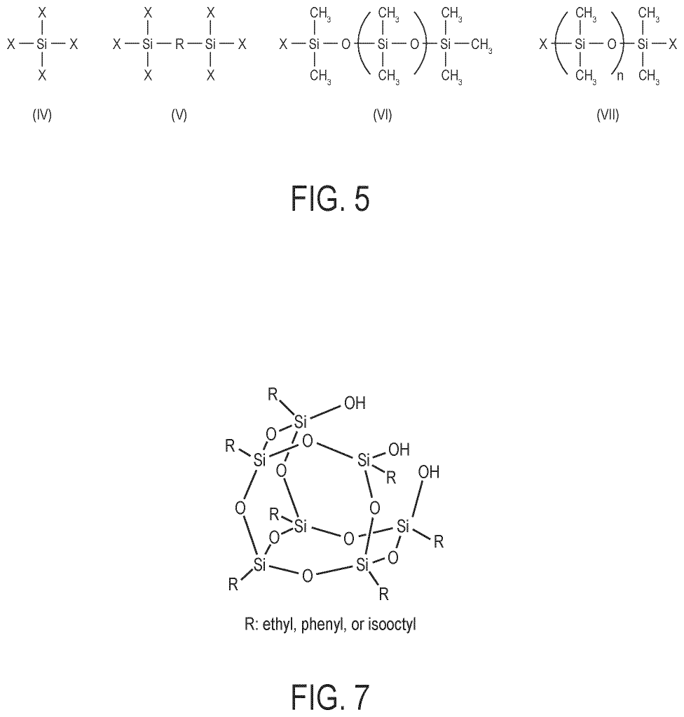

The crosslink density of the resultant organosilane coating film material would affect surface lubricity of material, in such a way that a greater crosslink density results in greater surface lubricity. The crosslink density may be increased by adding small amounts of additives. In some embodiments, the additives are hydrophobic monomeric silanes having four hydrolyzable moieties (X) per Formula (IV) shown in FIG. 5, e.g. tetraacetoxysilane, tetramethoxysilane, tetraethoxysilane, etc. In other embodiments, the additives are hydrophobic dipodal silanes per Formula (V) shown in FIG. 5, e.g. 1,6-bis(trimethoxysilyl)hexane, 1,2-bis(triethoxysilyl)ethane, bis(trimethoxysilylethyl)benzene, etc. Yet another embodiment utilizes a combination thereof.

In contrast, a lower crosslink density of the organosilane coating film material provides a more desirable material bulk modulus and acoustic impedance. The crosslink density may be reduced by utilizing minor monomeric organosilanes having two hydrolyzable moieties (X) per Formula (II) shown in FIG. 3 or one hydrolyzable moiety (X) per Formula (III) shown in FIG. 3. This generates linear or branched molecules covalently tethered to the primary crosslinked polymer network via siloxane bonds for the resultant organosilane coating film material. Similarly, to decrease the crosslink density and thus improve material flexibility for resultant organosilane coating film material, oligomeric polydimethylsiloxane (PDMS) having one to two terminal hydrolyzable groups (X), per Formula (VI) and (VII) of FIG. 5, may be also utilized.

One primary factor which contributes to the ability of a monomeric organosilane to generate a lubricious hydrophobic surface is its constituent organofunctional group, i.e. R moiety per Formula (I), (II), (III) and (V) as shown in FIG. 3. To impart coating hydrophobicity without impairing the hydrolysis-condensation reactivity, the organofunctional group (R) for the so-called hydrophobic organosilanes are generally selected from linear, branched and fluorinated alkyl groups and aryl groups, e.g. methyl, ethyl, propyl, n-butyl, t-butyl, i-butyl, phenyl, etc.

In some embodiments, the organosilane coating dispersion includes one or more organosilanes having one to three hydrolyzable acetoxy groups, such as Methyl triacetoxysilane, Ethyl triacetoxysilane, Vinyl triacetoxysilane, Phenyl triacetoxysilane, Methacryloxypropyl triacetoxysilane, Dimethyl diacetoxysilane, Vinylmethyl diacetoxysilane, Phenyldimethyl acetoxysilane, triethyl acetoxysilane, n-Butyldimethyl acetoxysilane, etc. In some embodiments, the organosilane coating dispersion includes one or more organosilanes having three hydrolyzable methoxy or chloro groups, such as Methyl trimethoxysilane, Ethyl trimethoxysilane, Ethyl trichlorosilane, etc. In some embodiments, the organosilane coating dispersion includes one or more performance additives, such as Tetraacetoxysilane, Tetramethoxysilane, Silanol-terminated PDMS, Silanol-functionalized POSS (polyhydral oligomeric silsesquioxane).

In addition, a polymer coating system or coating composition generally comprises one or more carrier solvents to dilute and disperse reactive organosilanes and other additives, such that the coating dispersion can be evenly applied to the substrate surface comprised of the silicone rubber material. To fabricate the lubricious, hydrophobic acoustic lens 146 without detrimentally affecting ultrasound imaging performances for the ICE ultrasound transducer, the integral concentration of reactive monomeric organosilanes and other reactive monomeric/oligomeric modifiers is controlled to be between 1 to 12% (v/v). In some embodiments, the concentration may be between 2 and 10% (v/v), and preferably between 2 and 8% (v/v). In some embodiments, the suitable carrier solvents of coating include any solvents in which constituent monomeric organosilanes and other modifiers of coating are soluble, or partially soluble, at ambient temperature. The selection of applicable carrier solvents or cosolvents closely depends on the types of constituent monomeric organosilanes and other modifiers as illustrated in FIGS. 3 and 5. The common examples of the carrier solvents may include a variety of volatile, nonpolar, hydrocarbon solvents, e.g. hexane, heptane, octane, cyclohexane, etc. for a hydrophobic organosilane coating formulation is essentially comprised of reactive monomeric organosilanes having hydrophobic organofunctional/hydrocarbon moieties (R). If R has some highly hydrophobic, fluorinated substitutions, some fluorinated cosolvent may be needed. In some embodiments where relevant coating formulation contains reactive monomeric organosilanes and modifiers having constituent acetoxy hydrolyzable moieties, the use of certain volatile ester solvents with mild polarity, e.g. methyl acetate, ethyl acetate, propyl acetate, isopropyl acetate, isobutyl acetate, tert-butyl acetate, ethyl acetoacetate, etc. is especially beneficial. In some embodiments where relevant coating formulation contains reactive monomeric organosilanes and modifiers having alkoxy hydrolyzable groups (e.g. methoxy and/or ethoxy), the use of certain volatile, polar alcohols, e.g. ethyl alcohol, n-propanol, isopropanol, n-butanol, etc. as the carrier cosolvents is preferred. In some embodiments, portions of nonpolar, volatile hydrocarbon solvent or silicone solvents comprised of cyclic and linear silicone molecules having a viscosity of less than 5 cSt (e.g. D4 to D6 cyclomethicones, dimethicones/linear PDMS, etc.) may be included into the organosilane coating formulations to activate and facilitate molecular penetration of reactive organosilanes and other modifiers into the subsurface of the silicone rubber material via solvent swelling, which may improve the layer-to-layer adherence and wear resistance for the ICE ultrasound transducer 110 comprising the lubricious acoustic lens 146.

Furthermore, the above carrier solvents may be mutually miscible in certain ratios due to their partial molecular similarity and small molecular sizes, and thus combinations thereof may be practically used as the carrier cosolvent system for formulating various organosilane coating compositions to attain optimal dissolution, dispersion, handling, and application of coating. For example, nonpolar hydrocarbons (e.g. hexane, heptane, etc.) exhibit excellent solubility with polar alcohols (e.g. ethyl alcohol, isopropanol, etc.), which in turn, have good solubility with mildly polar acetate solvents (e.g. ethyl acetate, t-butyl acetate). Also, nonpolar cyclomethicones and dimethicones possess good solubility in most of anhydrous alcohols having high polarity and hydrocarbons or halogenated hydrocarbons having nonpolarity.

Typically, silicone rubber is a difficult polymer material for application of a polymer coating of any kinds, for it inherently has very low surface energy (e.g. 19 to 22 mN/m or mJ/m²) and very high hydrophobicity (e.g. water contact angle of about 120°). Despite optimal use of different carrier solvents/co-solvents for coating formulation, resultant coating wettability onto the substrate surface of the silicone rubber material may be limited by considerable differences in surface tension between the coating dispersion and the silicone rubber substrate material. Such poor wettability would compromise coating adherence and give rise to rough surface topology with the presence of irregular coating droplets on coated surfaces. To overcome such technical challenges, in some embodiments, liquid wettability of the organosilane/silane coating formulations is tuned up by incorporating functional nanoparticles into relevant lubricious, hydrophobic organosilane coating compositions.

In addition, the incorporation of compatible nanoparticles or nanofillers into a superhydrophobic polymer coating is viable and effective for reducing the surface oleophilicity—the affinity of a surface to oily substances—of conventional hydrophobic organosilane/silane coating systems. Thus, to impart self-cleaning or anti-fouling capacity for organosilane coating and to enhance/maintain the surface lubricity and ultrasound imaging performances, superhydrophobic surfaces, such as nanoparticles or nanofiller, which are generally associated with the rough or patterned surfaces at a submicron or nanometer scale (i.e. the so-called lotus effect), are highly desirable for fabricating the ICE ultrasound transducer 110.

In some embodiments, applicable nanoparticles may be selected from hydrophilic fumed silica (or silicon dioxide) with or without surface treatment by so-called silane coupling agents that are chemically similar to constituent monomeric organosilanes of coating. Nanoparticle sizes may range from 1 to 100 nm, but preferably 1 to 50 nm. For example, fumed silica nanoparticles generally have particle sizes of about 5 to 50 nm (e.g. Aerosil® series fumed silica products commercially available from Evonik Inc.) and nonporous silicon dioxide nanoparticles having particle sizes of 10 to 30 nm and with/without specialty surface treatment (which are commercially available from SkySpring nanomaterials Inc.) can be utilized for the purposes. Alternatively, polyhedral oligomeric silsesquioxane (POSS) nanoparticles having the same or similar organofunctional moiety (R) and/or hydrolyzable moieties (X) to the constituent monomeric organosilanes and monomeric/oligomeric modifiers/additives, having a size of 1 to 3 nm, can be also considered, and such POSS nanoparticles are commercially available from Hybrid Plastics. These silicon-containing nanoparticles, including silica and POSS, are preferred, for they have inherent compatibility and affinity with the silicone rubber material and the cured organosilane coating materials comprised of the crosslinked siloxane polymer network. Alternatively, other hydrophilic inorganic nanoparticles, such as titanium dioxide, barium sulfate, etc., which are commonly available from many nanomaterial suppliers, such as Cerion Nanomaterials, SkySpring Nanomaterials Inc., etc. may also be used. These hydrophilic nanoparticles can have interfacial condensation reaction with the constituent monomeric organosilane ingredients of coating during the curing of coating or can be optionally be pre-modified by similar or same silane or organosilane agent as any of the constituent monomeric organosilane ingredients comprised of the nanocoating formulation, thus providing good compatibility with the coating compositions.

In some embodiments, lubricious nanocoating dispersions or formulations may contain a condensation catalyst to expedite relevant crosslinking cure reactions for the above nanocoating dispersions. Such catalysts are known to those skilled in the art, and may be selected from tin- or titanium-based organometallic compounds, such as dibutyltin diacetate, stannous octoate, dibutyltin dilaurate, etc. The amount of the catalyst used in the above nanocoating dispersion may be in the range of 0.01 to 10.00%, preferably 0.05 to 5.00%, with respect to the total organosilane reactants.

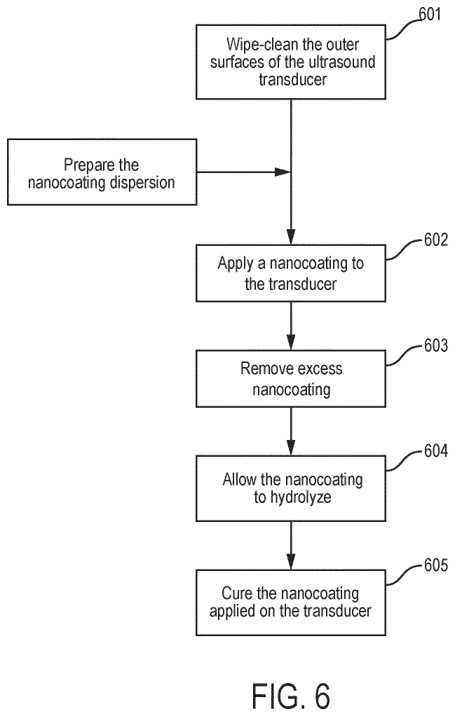

The organosilane nanocoating dispersion as discussed above may be applied to the entire surfaces of the outer polymeric encapsulant layer 140 comprising the matching material layer 144 of the ultrasound transducer 110. FIG. 6 provides a process flow chart of a coating method for the relevant embodiments disclosed above. Prior to coating, in step 601, the surface of the ultrasound transducer 110 to be coated are thoroughly wipe-cleaned, preferably using a lint-free cloth wetted by acetone, 100% IPA, dimethicone, or cyclomethicone solvent, such that the surface is free of any particulates or fibrous foreign materials. In step 602, the surface of the ultrasound transducer 110 is coated with the nanocoating dispersion. In some embodiments, coating may be performed by dip coating or spray coating. For dip coating, the cleaned ultrasound transducer surface to be coated is fully immersed into the nanocoating dispersion for 1 to 10 minutes, preferably 1 to 2 minutes, to attain the desirable coating thickness. Alternatively, for spray coating, one or more passes of spraying may be evenly applied to the surface of the ultrasound transducer 110 using a liquid spray system (e.g. DeVilbiss EGA503390F EGA). To remove excess coating and obtain an even liquid film of coating on the coated surface of the ultrasound transducer 110, in Step 603, the coated ultrasound transducer 110 is then briskly immersed into an aqueous solution (e.g. 50% isopropanol, 50% ethyl acetate, or 10% acetic acid, etc.) for about 1 to 20 seconds, preferably 1 to 5 seconds. In some embodiments, the aqueous solution comprises at least one of an alcohol, an ester, or a carboxylic acid, in a concentration of 10% to 90% (v/v) and preferably 40 to 60% (v/v). By briefly exposing to such water-containing solvent, the liquid film of coating evenly coated on the transducer surface at rest would undertake hydrolysis, in Step 604, under ambient condition. In some embodiments, the rest time to allow for hydrolysis is about 10 to 60 minutes, and preferably for 10 to 20 minutes. Thereafter, in step 605, the coated ultrasound transducer is allowed to slowly cure under dry, ambient environment, or quickly cured in a convective or vacuum oven at an elevated cure temperature. The elevated cure temperature is selected primarily based on the safety consideration of protecting the ultrasound transducer comprising the delicate electrical circuitries (e.g. PCB 116 and ASIC 114) from any thermal damage, and ranges from 40 to 90° C., preferably 55 to 70° C.

Examples set forth below in Table 1 give some examples of lubricious organosilane nanocoating dispersions (or formulations) based on certain embodiments as disclosed above. Liquid constituent ingredients (including organosilanes, catalysts, carrier solvents, etc.) used thereby are commercially available from one or more vendors, e.g. MilliporeSigma, Dow Corning, Gelest, Evonik, Wacker, etc. Specialty nanoparticle ingredients are also available from multiple vendors, including but not limited to, SkySpring Nanomaterials, Evonik, Hybrid Plastics, etc. Typical organosilane nanocoating dispersions, per certain embodiments as disclosed above, are prepared, first by sequentially adding various liquid constituent ingredients (including organosilanes, catalysts, carrier solvents, etc.) as measured in pertinent proportions by using proper pipettes, and then by adding constituent nanoparticles pre-weighed using a high-precision analytical balance.

TABLE 1
Exemplary organosilane nanocoating dispersions

| No. | Ingredients | | |
|---|---|---|---|
| #1 | Heptane (cosolvent) | 5 | mL |
| | Ethyl acetate (solvent) | 15 | mL |
| | Methyl triacetoxysilane (M-TAS) | 1.0 | mL |
| | Ethyl triacetoxysilane (E-TAS) | 0.5 | mL |
| #2 | Heptane (cosolvent) | 5 | mL |
| | Ethyl acetate (cosolvent) | 15 | mL |
| | Methyl triacetoxysilane (M-TAS) | 1.0 | mL |
| | Ethyl triacetoxysilane (E-TAS) | 0.5 | mL |
| | Dibutyl diacetate (catalyst) | 0.03 | mL |
| | Silica nanoparticle (6851HN) | 0.1 | g |
| #3 | Heptane (cosolvent) | 5 | mL |
| | Ethyl acetate (solvent) | 15 | mL |
| | Methyltriacetoxysilane (M-TAS) | 1.0 | mL |
| | Ethyl triacetoxysilane (E-TAS) | 0.5 | mL |
| | Dibutyl diacetate (catalyst) | 0.03 | mL |
| | Silica nanoparticle (Aerosil R202) | 0.1 | g |
| #4 | Heptane (cosolvent) | 5 | mL |
| | Ethyl acetate (solvent) | 15 | mL |
| | Methyl triacetoxysilane (M-TAS) | 1.5 | mL |
| | Dibutyl diacetate (catalyst) | 0.03 | mL |
| | POSS nanoparticle (SO1450) | 0.1 | g |
| #5 | Heptane (cosolvent) | 7.5 | mL |
| | Ethyl acetate (solvent) | 12.5 | mL |
| | Ethyl triacetoxysilane (M-TAS) | 1.5 | mL |
| | Dibutyl diacetate (catalyst) | 0.03 | mL |
| | Silica nanoparticles (6811DL) | 0.1 | g |
| #6 | Heptane (cosolvent) | 7.5 | mL |
| | Ethyl acetate (solvent) | 12.5 | mL |
| | Tetraacetoxysilane (TTAS) | 1.5 | g |
| | Dibutyl diacetate (catalyst) | 0.03 | mL |
| | Silica nanoparticle (6811DL) | 0.1 | g |
| #7 | Heptane (cosolvent) | 7.5 | mL |
| | Ethyl acetate (solvent) | 12.5 | mL |
| | Tetraacetoxysilane (TTAS) | 0.5 | mL |
| | Methyl trimethoxysilane (M-TMS) | 1.0 | mL |
| | Dibutyl diacetate (catalyst) | 0.03 | mL |
| | Silica nanoparticles (6811DL) | 0.1 | g |
| #8 | Heptane (cosolvent) | 7.5 | mL |
| | Ethyl acetate (solvent) | 12.5 | mL |
| | Tetraacetoxysilane (TTAS) | 0.5 | mL |
| | Ethyl trimethoxysilane (E-TMS) | 1.0 | mL |
| | Dibutyl diacetate (catalyst) | 0.03 | mL |
| | Silica nanoparticles (6811DL) | 0.1 | g |
| #9 | Heptane (cosolvent) | 7.5 | mL |
| | Ethyl acetate (cosolvent) | 12.5 | mL |
| | Tetraacetoxysilane (TTAS) | 0.5 | g |
| | Ethyl trichlorosilane (E-TClS) | 1.0 | mL |
| | Silica nanoparticles (6851HN) | 0.1 | g |
| #10 | Heptane (cosolvent) | 7.5 | mL |
| | Ethyl acetate (cosolvent) | 12.5 | mL |
| | Ethyl triacetoxysilane (E-TAS) | 1.2 | mL |
| | Silanol-terminated PDMS (DMS-S21) | 0.3 | mL |
| | Dibutyl diacetate (catalyst) | 0.03 | mL |
| | Silica nanoparticles (6811DL) | 0.1 | g |
| #11 | Heptane (cosolvent) | 7.5 | mL |
| | Ethyl acetate (cosolvent) | 12.5 | mL |
| | Methyl triacetoxysilane (M-TAS) | 1.2 | mL |
| | Silanol-functionalized POSS | 0.3 | mL |
| | Dibutyl diacetate (catalyst) | 0.03 | mL |
| | Silica nanoparticles (6811DL) | 0.1 | g |
| #12 | Heptane (cosolvent) | 7.5 | mL |
| | Ethyl acetate (cosolvent) | 12.5 | mL |
| | Ethyl triacetoxysilane (E-TAS) | 1.2 | mL |
| | Vinyl triacetoxysilane (V-TAS) | 0.3 | mL |
| | Dibutyl diacetate (catalyst) | 0.03 | mL |
| | Thermal or UV Initiator | 0.3 | mL |
| | Silica nanoparticles (6853HN) | 0.1 | g |

To exemplify the benefit of the nanoparticles, formulation #1 is a typical conventional organosilane coating dispersion without inclusion of nanoparticles or other performance additives. It is based on two monomeric organosilanes: methyl triacetoxysilane (M-TAS) and ethyl triacetoxysilane (E-TAS). When applied to the silicone rubber substrate (or the ultrasound transducer 110) and cured, this coating dispersion can result in the formation of the hydrophobic, lubricious coating material adherently bonded onto the silicone rubber substrate. However, poor wettability of the coating dispersion onto the substrate may create macroscopically uneven coating surfaces that may compromise surface lubricity and abrasive resistances of the coating film material, such that it may not be optimally suitable to the application of the ultrasound transducer 110 comprising the acoustic lens 146, because the conventional coating film material is largely lacking superhydrophobicity and self-cleaning performances.

Formulation #2, #3 and #4, as compared to Formulation #1, incorporate different silicon-containing nanoparticles, namely SkySpring's 6851HN and Evonik's Aerosil R202 with similar primary particle sizes of 10 to 25 nm and Hybrid Plastics' SO1450 with primary particle sizes of 1 to 3 nm, respectively. The 6851HN silica nanoparticles are surface-modified with a silane coupling agent comprising amino group, while Aerosil R202 is surface-treated with polydimethylsiloxane. SO1450 POSS nanoparticles comprise multiple isobutyl and silanol functional groups. These nanoparticles have inherent chemical compatibility with the constituent organosilane ingredients of the coating dispersion (i.e. Formulation #1) and improve liquid wettability of nanocoating on the silicone rubber substrate due to the formation of polymeric/oligomeric canopies, and then impart superhydrophobicity because of evenly-distributed nano-sized surface texture for the resultant organosilane nanocoating film material inherently adherent to the silicone rubber substrate surface. In addition, the amino functional group as provided by the surface-treated silica nanoparticle (i.e. 6851HN) or the constituent multi-silanol functional group of the POSS nanoparticle (i.e. SO1450) would promote the underlying cure reactions and thus enhance crosslink density for improving surface lubricity of coating. After curing, the modified surface 148 of the ultrasound transducer 110, including the formed acoustic lens 146, exhibits superhydrophobicity and self-cleaning, surface lubricity, and inherent adhesion to the outer polymeric encapsulant layer 140.

Formulations #5 through #12 incorporate the same surface-treated silica nanoparticle (i.e. 6811DL), but use different organosilane ingredients for the purpose of balancing the performance attributes of the acoustic lens (e.g. superhydrophobicity and surface lubricity) and/or imparting secondary UV/light curability of coating as well, etc.

Formulation #5, uses only E-TAS without use of M-TAS. This may improve the superhydrophobicity of the resultant acoustic lens because of more hydrophobic R-functional groups (i.e. R: ethyl versus methyl) comprised of the crosslinked siloxane polymer matrix (see FIG. 4).

The use of tetraacetoxysilane (TTAS) in Formulation #6 provides more crosslinkable sites for relevant silane nanocoating dispersion per the condensation cure reaction of hydrolysable silanes. The resultant nanocoating film material or acoustic lens would tend to exhibit the highest crosslink density and thus the highest surface lubricity and mechanical rigidity. To attain the balanced acoustic properties such as superhydrophobicity and mechanical properties, TTAS can be used along with M-TAS (e.g. Formulation #7), or E-TAS (e.g. Formulation #8), or other organosilane ingredient (e.g. ethyltrichlorosilane for Formulation #9), in proper proportions.

In any cases when good mechanical flexibility is essentially required for making the lubricious acoustic lens with the balanced acoustic impedance and superhydrophobicity, a silanol-terminated PDMS oligomer having a definite molecular weight, along with other monomeric ingredient (such as M-TAS or E-TAS), can be utilized as one of organosilane ingredients for formulating the organosilane nanocoating dispersion (e.g. Formulation #10). This will reduce crosslink density of coating and result in the flexible acoustic lens having the desirable acoustic impedance, while surface lubricity and superhydrophobicity can be largely maintained. In contrast, in any cases when high mechanical rigidity is essentially required for making the lubricious acoustic lens with the enhanced acoustic impedance and superhydrophobicity, a functionalized POSS (i.e. poly(hedral oligomeric silsesquoxane)) comprising trisilanol group, e.g. Hybrid Plastics' SO1450, SO1455, SO-1458, etc., as chemically illustrated in FIG. 7, may be added to relevant organosilane nanocoating dispersion with, or without, use of any other nanoparticles. Such silanol-functionalized POSS is inherently compatible with the organosilane monomers/oligomers and provides reactive sites for the underlying condensation cure reaction of coating, while rigid POSS cages act as fully-encapsulated nanoparticles for imparting liquid wettability of nanocoating and result in superhydrophobicity and surface lubricity for the resultant acoustic lens comprised of the POSS cage-interconnected, crosslinked siloxane polymer network. Furthermore, such acoustic lens 146 and the coated surface 148 of the ultrasound transducer 110 would exhibit high mechanical rigidity and improved acoustic impendence due to reinforcing effects arisen from the interconnection of nano-sized POSS cages within the siloxane polymer matrix.

Based on the above embodiments, the underlying condensation cure reactions among the same or different organosilane ingredients lead to crosslinking and formation of the siloxane polymer network matrix chemically comprised of the acoustic lens 146 and the coated surface 148 of the ultrasound transducer 110, and such reactions can be thermally accelerated by exposing the coated acoustic transducer to an elevated temperature up to 70 C. Furthermore, such crosslinking reaction can be considerably enhanced by utilizing the special organosilane ingredient(s) comprising one or more UV-curable, unsaturated R-functional groups, including vinyl (e.g. Formulation #12), acryloyloxy, methacryloyloxy groups, etc., at which thermally activated or photoinitiated radical polymerization and crosslinking reactions can take place. This may considerably reduce relevant cure time in attaining the solidification of nanocoating and allow to move to next manufacturing steps in short time with the continuous or concurrent condensation cure reaction until completion. To activate the secondary UV/light curability of radical polymerization and crosslinking, suitable initiator, such as UV photoinitiator, Irgacure 369 or DBMP (i.e. 2-Benzyl-2-(dimethylamino)-4?-morpholinobutyrophenone), Irgacure 2959 or HMPP (i.e. 2-Hydroxy-2-methylpropiophenone), etc., is needed to add to relevant organosilane nanocoating formulation.

According to the above examples, various organosilane or silane nanocoating dispersions can be formulated and applied to the ultrasound transducer 110 comprising the outer polymeric encapsulant layer 140 cast-molded of the silicone rubber material. The nanocoating dispersion may be cured and chemically converted as the nanoparticle-filled, crosslinked siloxane polymer film material, or the acoustic lens 146 and the lubricious surface 148 of the ultrasound transducer 110, per the primary hydrolysis-condensation cure mechanism of hydrolysable silanes and/or the secondary cure mechanism of radical polymerization and crosslinking. The resultant acoustic lens 146 and the lubricious surface 148 of the ultrasound transducer 110 is thin in thickness at micron or submicron scale, and exhibits various desirable surface property and performance attributes, including surface lubricity, superhydrophobicity and self-cleaning, etc. These attributes can be synergistically controlled or balanced by utilizing different organosilane ingredients, functionalized PDMS oligomers of different molecular weights, and nanoparticles of different types and sizes, etc, as described with reference to the exemplary formulations of Table 1.

Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

What is claimed is:

1. An intracardiac echocardiography catheter, the catheter comprising:
a shaft; and
an ultrasound transducer at a distal end of the shaft, the ultrasound transducer comprising:
an outer polymeric encapsulant layer; and
a nanocoating applied to the outer polymeric encapsulant layer and configured to provide increased surface lubricity and self-cleaning properties to the ultrasound transducer.

2. The intracardiac echocardiography catheter of claim 1, the ultrasound transducer further comprising an acoustic stack, wherein a portion of the outer polymeric encapsulant layer above the acoustic stack forms a matching material layer and a portion of the nanocoating applied above the matching material layer forms an acoustic lens.

3. The intracardiac echocardiography catheter of claim 1, wherein the outer polymeric encapsulant layer comprises a silicone rubber or urethane rubber material.

4. The intracardiac echocardiography catheter of claim 1, wherein the nanocoating comprises a lubricious, superhydrophobic polymer coating.

5. The intracardiac echocardiography catheter of claim 4, wherein the nanocoating is an organosilane coating dispersion with silica nanoparticles.

6. The intracardiac echocardiography catheter of claim 5, wherein the organosilane coating dispersion comprises one or more organosilanes having one to three hydrolyzable acetoxy groups.

7. The intracardiac echocardiography catheter of claim 6, wherein the one or more organosilanes is selected from a group of consisting of: Methyl triacetoxysilane, Ethyl triacetoxysilane, Vinyl triacetoxysilane, Phenyl triacetoxysilane, Methacryloxypropyl triacetoxysilane, Dimethyl diacetoxysilane, Vinylmethyl diacetoxysilane, Phenyldimethyl acetoxysilane, triethyl acetoxysilane, and n-Butyldimethyl acetoxysilane.

8. The intracardiac echocardiography catheter of claim 5, wherein the organosilane coating dispersion comprises one or more organosilanes having three hydrolyzable methoxy or chloro groups.

9. The intracardiac echocardiography catheter of claim 8, wherein the one or more organosilanes is selected from a group of consisting of: Methyl trimethoxysilane, Ethyl trimethoxysilane, and Ethyl trichlorosilane.

10. The intracardiac echocardiography catheter of claim 5, wherein the organosilane coating dispersion comprises one or more performance additives from a group consisting of: Tetraacetoxysilane, Tetramethoxysilane, Silanol-terminated PDMS, and Silanol-functionalized POSS (polyhydral oligomeric silsesquioxane) nanoparticles.

11. An organosilane nanocoating dispersion for coating an intracardiac echocardiography ultrasound transducer, the dispersion comprising:
at least one solvent;
at least one reactive organosilane; and
at least one nano-sized particle substance.

12. The organosilane nanocoating dispersion of claim 11, further comprising at least one catalyst.

13. The organosilane nanocoating dispersion of claim 11, wherein the organosilane and nano-sized particle substance are selected to modify an outer surface of the ultrasound transducer to provide increased surface lubricity and self-cleaning properties.

14. The organosilane nanocoating dispersion of claim 11, wherein the at least one organosilane is selected from a group consisting of: Methyl triacetoxysilane, Ethyl triacetoxysilane, Vinyl triacetoxysilane, Phenyl triacetoxysilane, Methacryloxypropyl triacetoxysilane, Dimethyl diacetoxysilane, Vinylmethyl diacetoxysilane, Phenyldimethyl acetoxysilane, triethyl acetoxysilane, n-Butyldimethyl acetoxysilane, Methyl trimethoxysilane, Ethyl trimethoxysilane, and Ethyl trichlorosilane.

15. The organosilane nanocoating dispersion of claim 11, wherein the nano-sized particle substance comprises silicon-containing nanoparticles having a diameter between 1 to 50 nm.

16. The organosilane nanocoating dispersion of claim 15, wherein the silicon-containing nanoparticles are silica nanoparticles comprising a surface treatment by a silane coupling agent.

17. The organosilane nanocoating dispersion of claim 15, wherein the silicon-containing nanoparticles are POSS nanoparticles comprising one or more silanol functional groups.

18. The organosilane nanocoating dispersion of claim 11, wherein a concentration of the organosilanes in the dispersion is between 2 to 8% (v/v).

19. The organosilane nanocoating dispersion of claim 11, further comprising a thermal or UV initiator.

20. The organosilane nanocoating dispersion of claim 11, wherein the at least one solvent is selected from a group of solvents consisting of: hydrocarbon, ester, alcohol, acetone, cyclomethicone, and dimethicone; and wherein the at least one catalyst is a tin-based or titanium-based organometallic compound.

21. A method of manufacturing an ultrasound transducer, the method comprising:
manufacturing an ultrasound transducer comprising an outer polymeric encapsulant layer;
cleaning an outer surface of the ultrasound transducer;
applying a nanocoating to the ultrasound transducer;
removing excess nanocoating from the ultrasound transducer with an aqueous solution;
allowing the nanocoating applied on the ultrasound transducer to hydrolyze; and
curing the nanocoating on the ultrasound transducer.

22. The method of claim 21, wherein the outer polymeric encapsulant layer comprises a silicone rubber.

23. The method of claim 21, wherein applying the nanocoating to the ultrasound transducer comprises coating the ultrasound transducer using at least one of a dip coating process or a spray coating process.

24. The method of claim 21, wherein an aqueous solution comprises at least one of an alcohol, or an ester, or a carboxylic acid, in a concentration of 40 to 60% (v/v).

25. The method of claim 21, wherein the nanocoating applied on the ultrasound transducer is allowed to hydrolyze for 10 to 30 minutes.

26. The method of claim 21, wherein curing the nanocoating comprises allowing the coated ultrasound transducer to cure in a dry, ambient environment.

27. The method of claim 21, wherein curing the nanocoating comprises accelerated curing of the coated ultrasound transducer in a convective or vacuum oven at an elevated temperature, wherein the elevated cure temperature is a temperature at which the ultrasound transducer does not incur thermal damage.

28. The method of claim 27, wherein the elevated cure temperature is between 55 and 70° C.
